(19) United States
(12) Patent Application Publication
(10) Pub. No.: US 2009/0182254 A1
(43) Pub. Date: Jul. 16, 2009

(54) ORTHOPEDIC BRACE OF KNEE JOINT

(76) Inventor: Sang deog CHO, SEOUL (KR)

Correspondence Address: IPLA P.A., 3580 WILSHIRE BLVD., 17TH FLOOR, LOS ANGELES, CA, 90010, US

(21) Appl. No.: 12/189,108

(22) Filed: Aug. 8, 2008

(30) Foreign Application Priority Data
Jan. 11, 2008 (KR) 10-2008-0003288

(51) Int. Cl. A61F 5/00

(52) U.S. Cl. 602/26; 602/16; 602/36

(57) ABSTRACT

An orthopedic brace of a knee joint is disclosed. The orthopedic brace including a hinge assembly connecting a lower support member and an upper support member to have a certain relative angle, wherein the hinge assembly includes a first link hinged to an upper portion of the lower support member and a lower portion of the upper support member, a second link hinged to the lower support member at a position spaced from the first link and having a linear slit and a curved slit spaced from the linear slit, a first hinge connecting the lower support member to the second link, a second hinge connecting the lower support member to the first link, a third hinge connecting the upper support member to the first link and slidably coupled to the linear slit, and a rotation connector coupled to the upper support member and slidably coupled to the curved slit.

### CROSS REFERENCE

This application claims foreign priority under Paris Convention and 35 U.S.C. §119 to Korean Patent Application No. 10-2008-0003288, filed Jan. 11, 2008 with the Korean Intellectual Property Office.

### BACKGROUND OF THE INVENTION

1. Field of the Invention

The present invention relates to an orthopedic brace of a knee joint, and more particularly to an orthopedic brace of a knee joint provided at a knee portion to relieve the pain and to provide stability and correction of the knee joint.

2. Description of the Related Art

Generally, a brace of a knee joint is provided at a knee portion of a patient who has undergone orthopedic operation or treatment of a knee joint, a physically disabled person and old and weak people to control and stabilize movement of a knee joint and to correct a bent joint. The brace of a knee joint is also used to prevent a recurrence of knee damage.

Particularly, a brace of a knee joint is provided for a bowlegged patient having the legs bent outward and the knees wide apart when he stands up with his legs together or a knock-kneed patient having the knees abnormally close together and the ankles wide apart when he stands up with his legs together. In this case, the brace of a knee joint pushes the thighbone away from the shinbone when a wearer stands up straight or stretches his legs such that the close knee bones can be normally spaced from each other without excessive pressure.

While the wearer with the knee joint brace stretches his legs, an upper support member fixed to the thighbone is pushed away from a lower support member fixed to the shinbone and, at the same time, the upper support member is rotated with respect to a hinge connecting portion for connecting the lower support member to the upper support member.

In a conventional brace of a knee joint, parts and an assembly for guiding a linear movement of the upper support member moving in a direction away from the lower support member are operated independently from parts and an assembly for guiding a rotation of the upper support member having a rotation angle displacement to the forward side of the wearer. Accordingly, moving paths of the linear movement and the rotation intersect each other and frequently disturb each other, thereby causing a problem that joint movement and walking of the wearer cannot be smoothly performed.

### SUMMARY OF THE INVENTION

Therefore, the present invention has been made in view of the above problems, and it is an object of the present invention to provide an orthopedic brace of a knee joint including an upper support member linearly moved and rotated with respect to a connecting portion with a lower support member and capable of guiding the linear movement and the rotation of the upper support member having connected displacements to mutually induce each other without intersecting each other, thereby enabling smooth joint movement and walking of the wearer.

In accordance with an aspect of the present invention, there is provided an orthopedic brace of a knee joint comprising a hinge assembly which connects a lower support member for supporting a calf and an upper support member for supporting a thigh to have a certain relative angle according to a movement state of a wearer, wherein the hinge assembly comprises: a first link hinged to an upper portion of the lower support member and a lower portion of the upper support member; a second link which is hinged to the lower support member at a position spaced from the first link and includes a linear slit formed in a linear shape at a portion connected to the upper support member and a curved slit formed in a curved shaped at a position spaced from the linear slit; a first hinge which connects the lower support member to the second link; a second hinge which connects the lower support member to the first link; a third hinge which connects the upper support member to the first link and is slidably coupled to the linear slit of the second link; and a rotation connector which is coupled to the upper support member and is slidably coupled to the curved slit of the second link.

Preferably, the first hinge, the second hinge, the third hinge and the rotation connector are sequentially positioned from bottom to top while the lower support member stands up straight and a relative angle between the upper support member and the lower support member ranges from 105° to 176°.

Preferably, the second link is bent outwardly from the second hinge at a portion adjacent to the second hinge and extending from the first hinge to the third hinge such that a moving path of the second link does not intersect the second hinge.

Preferably, the linear slit of the second link is formed to have a length corresponding to a linear displacement of the third hinge moving from the first hinge, the third hinge having a rotation displacement at the same distance from the second hinge.

Preferably, the curved slit of the second link is formed to be extended along a longitudinal direction of the linear slit and bent toward the linear slit with regard to both a linear displacement of the third hinge in the linear slit of the second link and a rotation displacement of the rotation connector rotated with respect to the third hinge.

In accordance with another aspect of the present invention, there is provided an orthopedic brace of a knee joint comprising a hinge assembly which connects a lower support member for supporting a calf and an upper support member for supporting a thigh to have a certain relative angle according to a movement state of a wearer, wherein the hinge assembly comprises: a lower leaf spring which is installed at an upper portion of the lower support member to provide an elastic displacement in a horizontal direction and includes a slit extending in a vertical direction; an upper leaf spring which is installed at a lower portion of the upper support member to provide an elastic displacement in a horizontal direction and includes a slit extending in a vertical direction; a lower spring connecting portion which is slidably coupled to the slit of the lower leaf spring and is fixed to a lower potion of a knee connection pad connected to a side portion of a knee of the wearer; and an upper spring connecting portion which is slidably coupled to the slit of the upper leaf spring and is fixed to an upper potion of the knee connection pad.

In accordance with a further aspect of the present invention, there is provided an orthopedic brace of a knee joint comprising first and second hinge assemblies, each hinge assembly connecting a lower support member for supporting a calf and an upper support member for supporting a thigh to have a certain relative angle according to a movement state of a wearer, wherein the first hinge assembly, installed at an inside portion (or an outside portion) of a knee of the wearer, comprises a first link hinged to an upper portion of the lower support member and a lower portion of the upper support member, a second link which is hinged to the lower support member at a position spaced from the first link and includes a linear slit formed in a linear shape at a portion connected to the upper support member and a curved slit formed in a curved shaped at a position spaced from the linear slit, a first hinge which connects the lower support member to the second link, a second hinge which connects the lower support member to the first link, a third hinge which connects the upper support member to the first link and is slidably coupled to the linear slit of the second link, and a rotation connector which is coupled to the upper support member and is slidably coupled to the curved slit of the second link; and wherein the second hinge assembly, installed at an outside portion (or an inside portion) of a knee of the wearer, comprises a lower leaf spring which is installed at an upper portion of the lower support member to provide an elastic displacement in a horizontal direction and includes a slit extending in a vertical direction, an upper leaf spring which is installed at a lower portion of the upper support member to provide an elastic displacement in a horizontal direction and includes a slit extending in a vertical direction, a lower spring connecting portion which is slidably coupled to the slit of the lower leaf spring and is fixed to a lower potion of a knee connection pad connected to a side portion of a knee of the wearer, and an upper spring connecting portion which is slidably coupled to the slit of the upper leaf spring and is fixed to an upper potion of the knee connection pad.

Preferably, the orthopedic brace of a knee joint further comprises a lower sponge having a size increasing from bottom to top in a vertical section and installed at an upper portion of the lower support member; and an upper sponge having a size increasing from top to bottom in a vertical section and installed at a lower portion of the upper support member.

As described above, according to the present invention, the linear slit and the curved slit of the second link are separately formed such that moving paths of the linear movement and the rotation do not intersect or overlap each other and are also connectedly extended such that the linear movement and the rotation induce each other. Accordingly, the linear movement and the curved movement of the upper support member, intersecting each other, can be efficiently induced and guided while the upper support member moves in connection with the first link with respect to the lower support member.

Thus, in knee joint restoration for a bowlegged or knock-kneed patient, when the hinge assembly having the second link is installed at an inside portion or an outside portion of knee bones having a small distance therebetween, the upper support member linearly moves in a direction away from the lower support member while interlocking with the forward rotation of the upper support member. As a result, the hinge assembly can correct a bent joint of the wearer to enable smooth joint movement and walking.

Further, in knee joint restoration for a bowlegged or knock-kneed patient, when the hinge assembly having the lower plate spring and the upper plate spring is installed at an outside portion or an inside portion of knee bones having an extended distance therebetween, the lower leaf spring and the upper leaf spring are elastically deformed. Accordingly, even though the hinge assembly is used for a patient having a large knee joint due to excessive deformation of knee bones, a specific connection pressure can be maintained without excessively pressing the knee.

Further, when the lower sponge having a size increasing from bottom to top and the upper sponge having a size increasing from top to bottom are respectively attached to the lower support member and the upper support member, a simple structure of a single support member can be manufactured instead of a conventional structure of double support members with a leaf spring coupled therebetween. Further, it is possible to improve adhesion between the lower and upper support members and the skin of the wearer and wearing comfort of the brace. Since the sponge can be deformed flexibly and elastically by a pressing force between the skin of the wearer and the support member, the upper support member can move more smoothly.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates a perspective view showing an orthopedic brace of a knee joint according to the first embodiment of the present invention;

FIG. 2 illustrates another used state of the orthopedic brace shown in FIG. 1;

FIG. 3 illustrates a side view of FIG. 1;

FIG. 4 illustrates a side view showing a state in which an upper support member of FIG. 3 is moved by a rotation angle displacement of 19°;

FIG. 5 illustrates a side view showing a state in which the upper support member of FIG. 3 is moved by a rotation angle displacement of 38°;

FIG. 6 illustrates a side view showing a state in which the upper support member of FIG. 3 is moved by a rotation angle displacement of 57°;

FIG. 7 illustrates a side view showing a state in which the upper support member of FIG. 3 is moved by a rotation angle displacement of 71°;

FIG. 8 illustrates side views of essential parts showing a process in which the state of FIG. 3 is changed to the state of FIG. 7;

FIG. 9 illustrates a front view of an orthopedic brace of a knee joint according to a second embodiment of the present invention;

FIGS. 10A and 10B illustrate front views of essential parts of a second hinge assembly of FIG. 9 in a used state; and

FIG. 11 schematically shows an installation state of a lower sponge and an upper sponge.

### DETAILED DESCRIPTION OF THE PREFERRED EMBODIMENTS

An orthopedic brace of a knee joint according to the present invention includes hinge assemblies 300 and 400 for connecting a lower support member 100 supporting a calf and an upper support member 200 supporting a thigh to have a certain relative angle according to a movement state of a wearer. A hinge assembly capable of smoothly guiding the linear movement and the rotation is referred to as a first hinge assembly 300. A hinge assembly having a structure movable flexibly and elastically in a lateral direction is referred to as a second hinge assembly 400.

Hereinafter, an orthopedic brace of a knee joint according to embodiments of the present invention will be described in detail. In a first embodiment, the first hinge assembly 300 is installed on each of the left and right sides of a knee joint (an inside portion and an outside portion of a knee joint of a right leg of the wearer). In a second embodiment, the first hinge assembly 300 and the second hinge assembly 400 are installed on the left and right sides of the knee joint.

First, the orthopedic brace of a knee joint according to the first embodiment of the present invention will be described in detail with reference to the accompanying drawings.

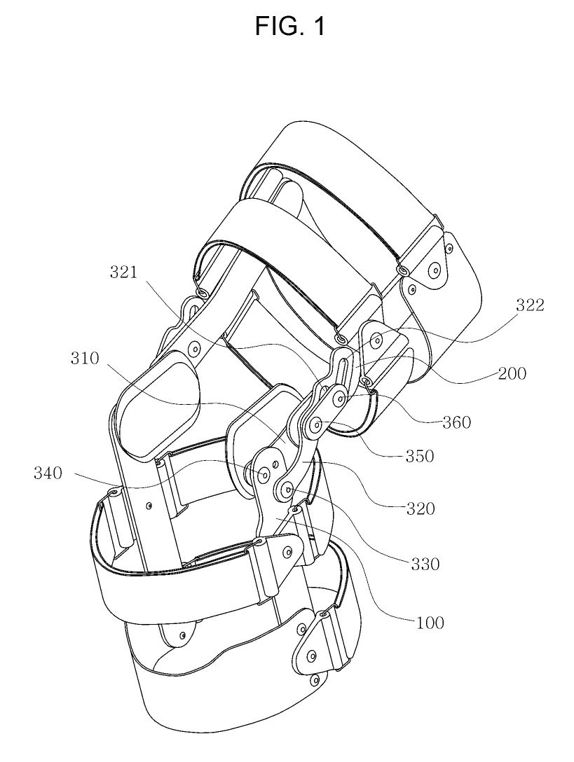

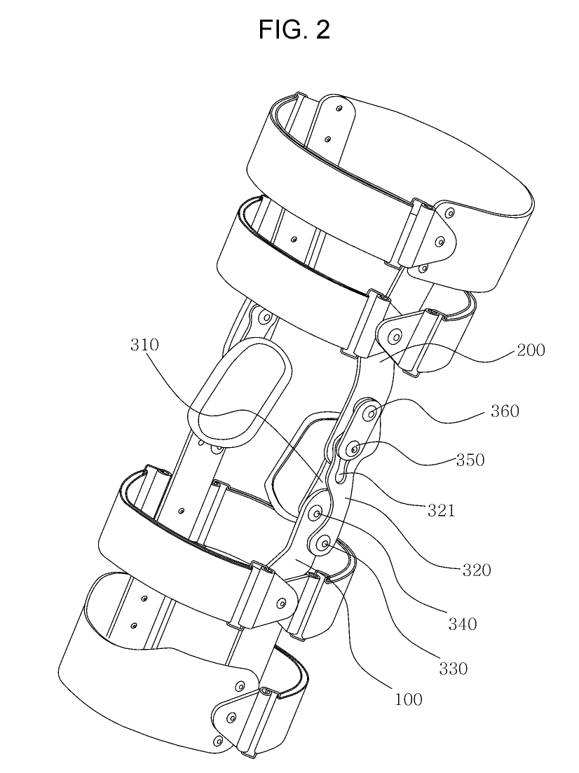

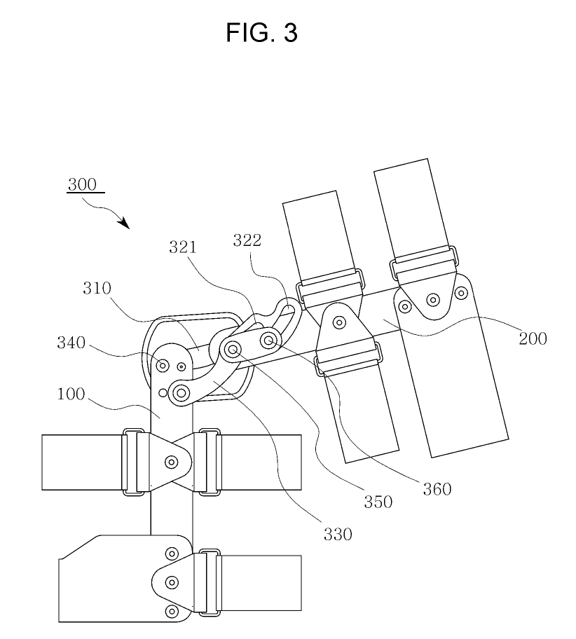

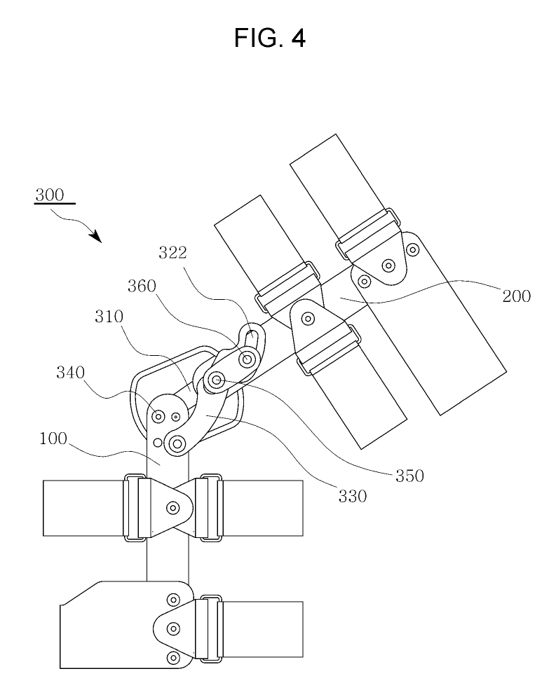

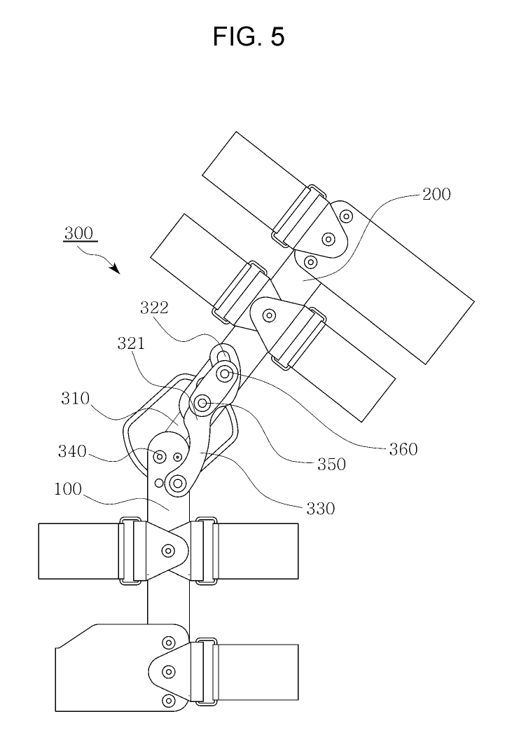

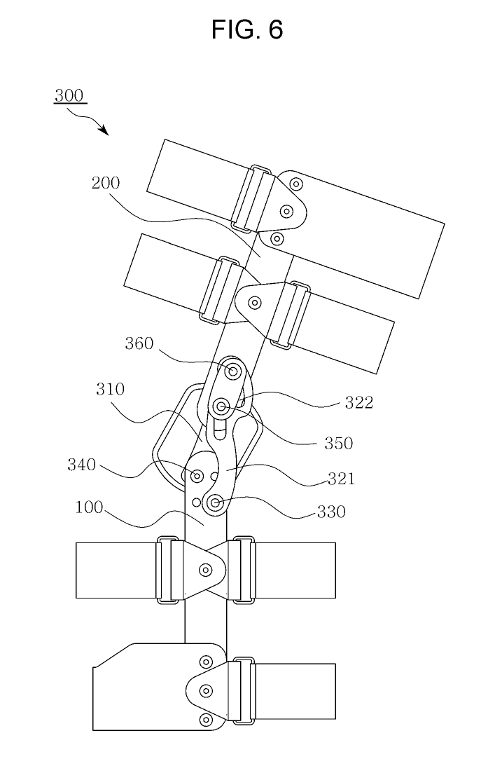

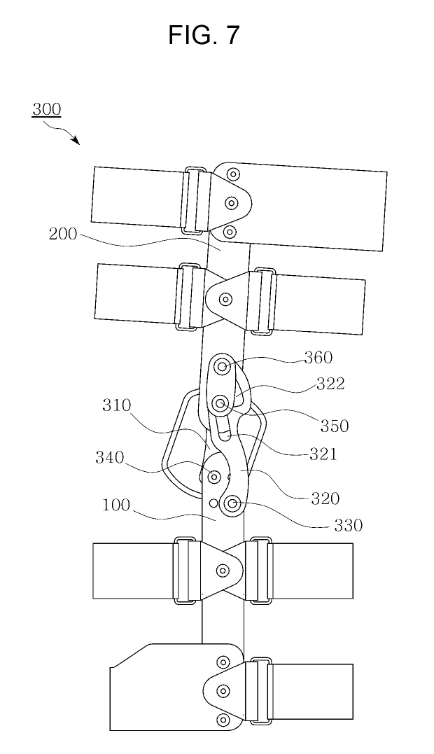

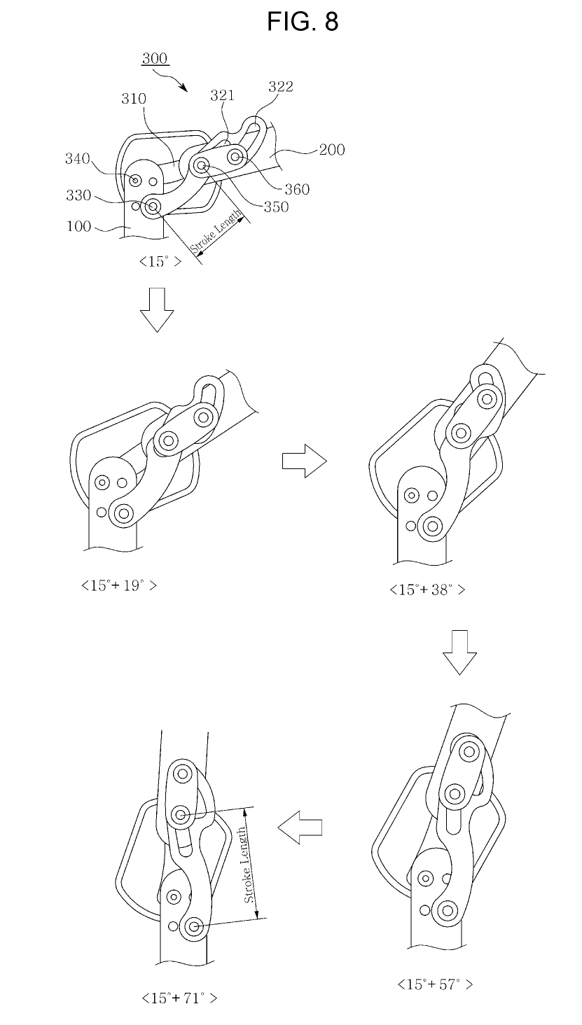

FIG. 1 illustrates a perspective view showing an orthopedic brace of a knee joint according to the first embodiment of the present invention. FIG. 2 illustrates another used state of the orthopedic brace shown in FIG. 1. FIG. 3 illustrates a side view of FIG. 1. FIGS. 4 to 7 illustrate side views showing the respective states in which the upper support member of FIG. 3 is moved by rotation angle displacements of 19°, 38°, 57° and 71°. FIG. 8 illustrates side views of essential parts showing a process in which the state of FIG. 3 is changed to the state of FIG. 7.

As shown in FIGS. 1 and 2, the first hinge assembly 300 includes a first link 310, a second link 320, a first hinge 330, a second hinge 340, a third hinge 350, and a rotation connector 360. The first hinge 330 connects the lower support member 100 with the second link 320, and the second hinge 340 connects the lower support member 100 with the first link 310. The third hinge 350 and the rotation connector 360 are coupled to the upper support member 200 to connect the upper support member 200 with the first link 310 while their portions are restricted by slits 321 and 322, respectively.

The first link 310 is installed between the lower support member 100 and the upper support member 200 to allow relative movement between the lower support member 100 and the upper support member 200. Lower and upper end portions of the first link 310 are hinged to an upper portion of the lower support member 100 and a lower portion of the upper support member 200, respectively. That is, the first link 310 is connected to the lower support member 100 by the second hinge 340, and the first link 310 is connected to the upper support member 200 by the third hinge 350.

The first link 310 separates the lower portion of the upper support member 200 from the upper portion of the lower support member 100 by a specific distance corresponding to a distance between the second hinge 340 and the third hinge 350 regardless of a moving state, a displacement and an angle of the upper support member 200.

The second link 320 optimizes the movement of the upper support member 200 when the upper support member 200 is rotated while being connected to the lower support member 100 by the first link 310. The second link 320 includes a linear slit 321 and a curved slit 322 capable of restricting a moving displacement of the upper support member 200 at a portion connected to the upper support member 200. Lower and upper end portions of the second link 320 are hinged to the upper portion of the lower support member 100 and the lower portion of the upper support member 200, respectively.

The second link 320 is hinged to the lower support member 100 by the first hinge 330 at a position spaced from the second hinge 340. The second link 320 includes the linear slit 321 and the curved slit 322 to allow the upper support member 200 to have a liner displacement and a curved displacement, respectively, with respect to the first hinge 330. Accordingly, the third hinge 350 and the rotation connector 360 are coupled to the upper support member 200 while their portions are restricted by the slits 321 and 322, respectively.

The first hinge 330 connecting the second link 320 to the lower support member 100 is installed at a position spaced from the second hinge 340 connecting the first link 310 to the lower support member 100. Accordingly, when the third hinge 350 connecting the first and second links 310 and 320 to the upper support member 200 is rotated with respect to the second hinge 340, the third hinge 350 is maintained at the same distance from the second hinge 340, whereas the third hinge 350 has a different distance from the first hinge 330 according to positions of the third hinge 350 and the upper support member 200. The linear slit 321 is formed to have a length corresponding to a reciprocating displacement of the third hinge 350 which moves toward or away from the first hinge 330.

The upper support member 200 can be freely rotated within a moving displacement formed by the linear slit 321 while its lower portion is hinged to the first and second links 310 and 320. Also, the upper support member 200 can be independently rotated with respect to the third hinge 350 regardless of a moving state and an extended direction of the first link 310 and the second link 320.

The curved slit 322 is formed to be extended along a longitudinal direction of the linear slit 321 and bent toward the linear slit 321 with regard to both a linear displacement of the third hinge 350 restricted by the linear slit 321 and a rotation displacement of the rotation connector 360 rotated with respect to the third hinge 350 positioned at a certain point in the linear slit 321.

The first hinge 330 connects the second link 320 to the lower support member 100, and the second hinge 340 connects the first link 310 to the lower support member 100. The third hinge 350 connects the first link 310 to the upper support member 200 while the third hinge 350 is slidably inserted into the linear slit 321 of the second link 320. The rotation connector 360 is coupled to the upper support member 200 while the rotation connector 360 is slidably inserted into the curved slit 322 of the second link 320.

The linear slit 321 is formed in a linear shape capable of smoothly guiding a linear movement of the third hinge 350 connected to the first and second links 310 and 320. The curved slit 322 guides a rotation of the rotation connector 360 and also upwardly moves the third hinge 350 maintained at the same distance from the rotation connector 360 when the upper support member 200 has a rotation angle displacement, thereby efficiently guiding an upward movement of the upper support member 200.

FIGS. 3 to 8 illustrate a process in which a relative angle between the lower support member 100 and the upper support member 200 is changed from 105° to 176°. The first hinge 330, the second hinge 340, the third hinge 350 and the rotation connector 360 are sequentially positioned from bottom to top to smoothly realize the above-described operation. Further, a coupling position between the second link 320 and the third hinge 350 and a coupling position between the second link 320 and the rotation connector 360 are changed according to the relative angle between the lower support member 100 and the upper support member 200.

FIG. 3 illustrates a state in which a relative angle of 105° is formed between the lower support member 100 and the upper support member 200. In the state of FIG. 3, the third hinge 350 and the rotation connector 360 are positioned at lower ends of the linear slit 321 and the curved slit 322 of the second link 320. While the relative angle is changed to 176°, as shown in FIGS. 4 to 7, the third hinge 350 and the rotation connector 360 are moved to upper ends of the linear slit 321 and the curved slit 322 of the second link 320 while the third hinge 350 and the rotation connector 360 are maintained at the same distance from each other.

In the orthopedic brace of a knee joint having the first hinge assembly 300 according to the present invention, the linear slit 321 and the curved slit 322 of the second link 320 are separately formed such that moving paths of the linear movement and the rotation of the upper support member 200 do not intersect or overlap each other. Further, the linear slit 321 and the curved slit 322 are connectedly extended such that the linear movement and the rotation induce each other. Accordingly, the linear movement and the curved movement of the upper support member 200, intersecting each other, are efficiently induced and guided while the upper support member 200 moves in connection with the first link 310 and the second link 320 with respect to the lower support member 100.

Thus, in knee joint restoration for a bowlegged or knock-kneed patient, when the first hinge assembly 300 is installed at an inside portion or an outside portion of knee bones having a small distance therebetween, the upper support member 200 linearly moves in a direction away from the lower support member 100 while interlocking with the forward rotation of the upper support member 200. As a result, the first hinge assembly 300 can correct a bent joint of the wearer to enable smooth joint movement and walking.

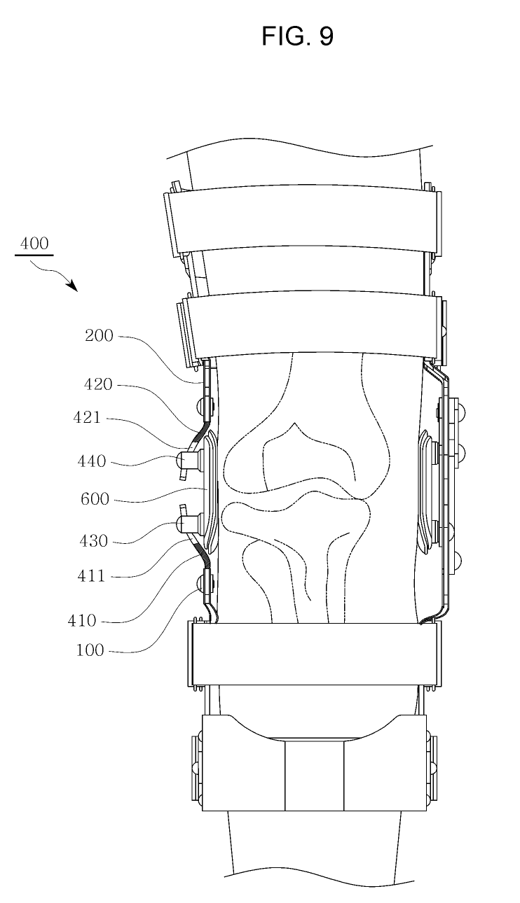

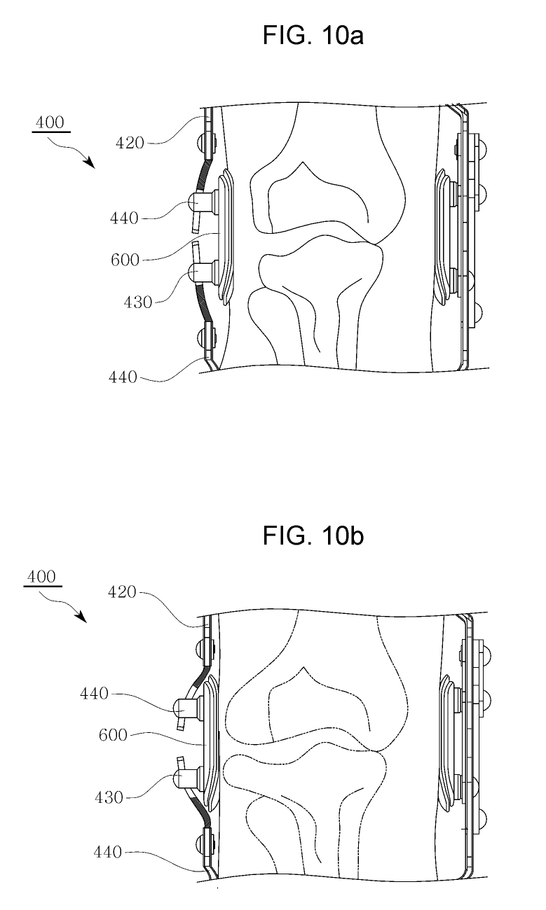

FIG. 9 illustrates a front view of an orthopedic brace of a knee joint according to a second embodiment of the present invention. FIGS. 10A and 10B illustrate front views of essential parts of a second hinge assembly of FIG. 9 in a used state.

As shown in FIG. 9, the orthopedic brace of a knee joint according to the second embodiment of the present invention includes the first hinge assembly 300 of the first embodiment installed on the left side of the knee joint required to be supported upward in a vertical direction when the wearer stands up straight, and the second hinge assembly 400 installed on the right side of the knee joint required to be variably extended in a horizontal direction.

The second hinge assembly 400 largely includes a lower leaf spring 410, an upper leaf spring 420, a lower spring connecting portion 430 and an upper spring connecting portion 440. The lower leaf spring 410 and the upper leaf spring 420 are coupled to the lower support member 100 and the upper support member 200, respectively. The lower spring connecting portion 430 and the upper spring connecting portion 440 are slidably coupled to the lower leaf spring 410 and the upper leaf spring 420.

The lower leaf spring 410 and the upper leaf spring 420 are installed at the upper portion of the lower support member 100 and the lower portion of the upper support member 200, respectively, to provide an elastic displacement in a horizontal direction. The adjacent end portions thereof are separated from a knee connection pad 600 connected to a side portion of the knee of the wearer by a certain distance such that each of the end portions can reach the knee connection pad 600. Slits 411 and 421 are formed to be extended in a vertical direction at portions connected with the knee connection pad 600.

The lower spring connecting portion 430 and the upper spring connecting portion 440 are slidably coupled to the slit 411 of the lower leaf spring 410 and the slit 421 of the upper leaf spring 420, respectively. The lower spring connecting portion 430 and the upper spring connecting portion 440 are fixed to a lower portion and an upper portion of the knee connection pad 600, respectively.

When a bowlegged or knock-kneed patient having extended and deformed knee bones wears an orthopedic brace of a knee joint having the second hinge assembly 400, as shown in FIGS. 10A and 10B, the end portions of the lower and upper leaf springs 410 and 420 are pushed in a horizontal direction according to a protruded state of the knee bones of the wearer, and the end portions are further spaced from each other within a limited moving range formed by the slits 411 and 421. The knee connection pad 600 is moved in a horizontal direction while being restricted by the lower and upper leaf springs 410 and 420 due to the lower and upper spring connecting portions 430 and 440.

In knee joint restoration for a bowlegged or knock-kneed patient, when the second hinge assembly 400 is installed at an outside portion or an inside portion of knee bones having an extended distance therebetween, the lower leaf spring 410 and the upper leaf spring 420 are elastically deformed. Accordingly, even though the second hinge assembly 400 is used for a patient having a large knee joint due to excessive deformation of knee bones, a specific connection pressure can be maintained without excessively pressing the knee.

Thus, when the first hinge assembly 300 is installed at an inside portion or an outside portion of knee bones having a small distance therebetween and the second hinge assembly 400 is installed at an outside portion or an inside portion of knee bones having an extended distance therebetween, the first hinge assembly 300 installed at one side can correct a bent joint of the wearer to enable smooth joint movement and walking and, at the same time, the second hinge assembly 400 installed at the other side can correct a knee joint with a specific connection pressure without excessively pressing the knee.

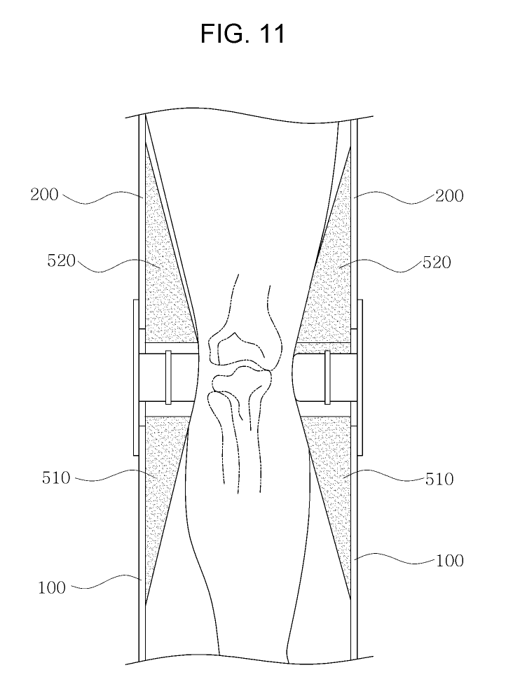

FIG. 11 schematically shows an installation state of a lower sponge and an upper sponge. In the application of the first and second embodiments, as shown in FIG. 11, it is preferable to additionally install a lower sponge 510 and an upper sponge 520 to the lower support member 100 and the upper support member 200 provided at a connection portion between a calf and a thigh. That is, the lower sponge 510 having a size increasing from bottom to top in the vertical section is installed at the upper portion of the lower support member 100, and the upper sponge 520 having a size increasing from top to bottom in the vertical section is installed at the lower portion of the upper support member 200.

When the lower sponge 510 and the upper sponge 520 are installed, it is possible to improve adhesion between the lower and upper support members 100 and 200 and the skin of the wearer and wearing comfort of the brace. Further, a simple structure of a single support member can be manufactured instead of a conventional structure of double support members with a leaf spring coupled therebetween. Since the sponge can be deformed flexibly and elastically by a pressing force between the skin of the wearer and the support member, the upper support member 200 can be more smoothly extended with respect to the first hinge 330.

Although the preferred embodiment of the present invention has been disclosed for illustrative purposes, those skilled in the art will appreciate that various modifications, additions and substitutions are possible, without departing from the scope and spirit of the invention as disclosed in the accompanying claims.

What is claimed is:

1. An orthopedic brace of a knee joint comprising a hinge assembly which connects a lower support member for supporting a calf and an upper support member for supporting a thigh to have a certain relative angle according to a movement state of a wearer, wherein the hinge assembly comprises:
a first link hinged to an upper portion of the lower support member and a lower portion of the upper support member;
a second link which is hinged to the lower support member at a position spaced from the first link and includes a linear slit formed in a linear shape at a portion connected to the upper support member and a curved slit formed in a curved shaped at a position spaced from the linear slit;
a first hinge which connects the lower support member to the second link;
a second hinge which connects the lower support member to the first link;
a third hinge which connects the upper support member to the first link and is slidably coupled to the linear slit of the second link; and
a rotation connector which is coupled to the upper support member and is slidably coupled to the curved slit of the second link.

2. The orthopedic brace of a knee joint according to claim 1, wherein the first hinge, the second hinge, the third hinge and the rotation connector are sequentially positioned from bottom to top while the lower support member stands up straight and a relative angle between the upper support member and the lower support member ranges from 105° to 176°.

3. The orthopedic brace of a knee joint according to claim 1, wherein the second link is bent outwardly from the second hinge at a portion adjacent to the second hinge and extending from the first hinge to the third hinge such that a moving path of the second link does not intersect the second hinge.

4. The orthopedic brace of a knee joint according to claim 2, wherein the second link is bent outwardly from the second hinge at a portion adjacent to the second hinge and extending from the first hinge to the third hinge such that a moving path of the second link does not intersect the second hinge.

5. The orthopedic brace of a knee joint according to claim 1, wherein the second link is bent outwardly from the second hinge at a portion adjacent to the second hinge and extending from the first hinge to the third hinge such that a moving path of the second link does not intersect the second hinge.

6. The orthopedic brace of a knee joint according to claim 2, wherein the linear slit of the second link is formed to have a length corresponding to a linear displacement of the third hinge moving from the first hinge, the third hinge having a rotation displacement at the same distance from the second hinge.

7. The orthopedic brace of a knee joint according to claim 1, wherein the curved slit of the second link is formed to be extended along a longitudinal direction of the linear slit and bent toward the linear slit with regard to both a linear displacement of the third hinge in the linear slit of the second link and a rotation displacement of the rotation connector rotated with respect to the third hinge.

8. An orthopedic brace of a knee joint comprising a hinge assembly which connects a lower support member for supporting a calf and an upper support member for supporting a thigh to have a certain relative angle according to a movement state of a wearer, wherein the hinge assembly comprises:
a lower leaf spring which is installed at an upper portion of the lower support member to provide an elastic displacement in a horizontal direction and includes a slit extending in a vertical direction;
an upper leaf spring which is installed at a lower portion of the upper support member to provide an elastic displacement in a horizontal direction and includes a slit extending in a vertical direction;
a lower spring connecting portion which is slidably coupled to the slit of the lower leaf spring and is fixed to a lower potion of a knee connection pad connected to a side portion of a knee of the wearer; and
an upper spring connecting portion which is slidably coupled to the slit of the upper leaf spring and is fixed to an upper potion of the knee connection pad.

9. An orthopedic brace of a knee joint comprising first and second hinge assemblies, each hinge assembly connecting a lower support member for supporting a calf and an upper support member for supporting a thigh to have a certain relative angle according to a movement state of a wearer, wherein the first hinge assembly, installed at an inside portion (or an outside portion) of a knee of the wearer, comprises a first link hinged to an upper portion of the lower support member and a lower portion of the upper support member, a second link which is hinged to the lower support member at a position spaced from the first link and includes a linear slit formed in a linear shape at a portion connected to the upper support member and a curved slit formed in a curved shaped at a position spaced from the linear slit, a first hinge which connects the lower support member to the second link, a second hinge which connects the lower support member to the first link, a third hinge which connects the upper support member to the first link and is slidably coupled to the linear slit of the second link, and a rotation connector which is coupled to the upper support member and is slidably coupled to the curved slit of the second link; and
wherein the second hinge assembly, installed at an outside portion (or an inside portion) of a knee of the wearer, comprises a lower leaf spring which is installed at an upper portion of the lower support member to provide an elastic displacement in a horizontal direction and includes a slit extending in a vertical direction, an upper leaf spring which is installed at a lower portion of the upper support member to provide an elastic displacement in a horizontal direction and includes a slit extending in a vertical direction, a lower spring connecting portion which is slidably coupled to the slit of the lower leaf spring and is fixed to a lower potion of a knee connection pad connected to a side portion of a knee of the wearer, and an upper spring connecting portion which is slidably coupled to the slit of the upper leaf spring and is fixed to an upper potion of the knee connection pad.

10. The orthopedic brace of a knee joint according to claim 9, further comprising:
a lower sponge having a size increasing from bottom to top in a vertical section and installed at an upper portion of the lower support member; and
an upper sponge having a size increasing from top to bottom in a vertical section and installed at a lower portion of the upper support member.
